(19) United States
(12) Patent Application Publication
(10) Pub. No.: US 2017/0112480 A9
(43) Pub. Date: Apr. 27, 2017

(54) EXPANDABLE DEVICE SHEATH FOR VASCULAR CLOSURE PLUG DEPLOYMENT

(71) Applicant: BOSTON SCIENTIFIC SCIMED, INC., Maple Grove, MN (US)

(72) Inventors: Mark L. Jenson, Greenfield, MN (US); Wayne M. Falk, Minneapolis, MN (US); Jason Hill, Brooklyn Park, MN (US); John Blix, Maple Grove, MN (US); William Drasler, Minnetonka, MN (US)

(73) Assignee: BOSTON SCIENTIFIC SCIMED, INC., Maple Grove, MN (US)

(21) Appl. No.: 13/677,422

(22) Filed: Nov. 15, 2012

Related U.S. Application Data
(63) Continuation-in-part of application No. 13/253,497, filed on Oct. 5, 2011, now Pat. No. 8,758,402.
(60) Provisional application No. 61/561,113, filed on Nov. 17, 2011.
(60) Provisional application No. 61/424,230, filed on Dec. 17, 2010.

(51) Int. Cl. A61B 17/00

(52) U.S. Cl. CPC A61B 17/0057

(57) ABSTRACT

The present disclosure relates generally to methods and devices for closing and/or sealing an opening in a vessel wall and/or an adjacent tissue tract. In some cases, the device includes a sheath having a distal end that can expand radially as needed. In some cases, the distal portion of the sheath is weakened so that it may tear in a predetermined manner. In some cases, the distal portion of the sheath is weakened so that it may deform plastically in a predetermined manner. In some cases, the distal portion of the sheath or the entire sheath may be folded so that it unfolds in a predetermined manner. In general, each of these sheaths may have a relatively small introduction profile, may have a distal portion that can expand to a larger diameter than the introduction profile during use, and can be collapsed again to a smaller profile for removal.

### CROSS-REFERENCE TO RELATED APPLICATIONS

This application claims the benefit of U.S. Provisional Application No. 61/561,113, filed Nov. 17, 2011, which is herein incorporated by reference.

### FIELD

The present disclosure relates generally to medical devices and more particularly to methods and devices for closing and/or sealing punctures in tissue.

### BACKGROUND

In many medical procedures, such as, for example, balloon angioplasty and the like, an opening can be created in a blood vessel or arteriotomy to allow for the insertion of various medical devices which can be navigated through the blood vessel to the site to be treated. For example, after initial access with a hollow needle, a guidewire may first be inserted through the tissue tract created between the skin, or the epidermis, of the patient down through the subcutaneous tissue and into the opening formed in the blood vessel. Once the guidewire is in place, an introducer sheath can be slid over the guide wire to form a wider, more easily accessible, tract between the epidermis and the opening into the blood vessel. The appropriate medical device can then be introduced through the introducer sheath and then up the blood vessel to the site of the occlusion or other treatment site.

Once the procedure is completed, the medical devices or other equipment introduced into the vessel can be retracted through the blood vessel, out the opening in the blood vessel wall, and out through the tissue tract to be removed from the body. The physician or other medical technician is presented with the challenge of trying to close the opening in the blood vessel and/or the tissue tract formed in the epidermis and subcutaneous tissue. A number of different device structures, assemblies, and methods are known for closing the opening in the blood vessel and/or tissue tract, each having certain advantages and disadvantages. However, there is an ongoing need to provide new and improved device structures, assemblies, and/or methods for closing and/or sealing the opening in the blood vessel and/or tissue tract.

### BRIEF SUMMARY

The following summary is provided to facilitate an understanding of some of the innovative features unique to the present disclosure and is not intended to be a full description. A full appreciation of the disclosure can be gained by taking the entire specification, claims, drawings, and abstract as a whole.

The present disclosure relates generally to medical devices and more particularly to methods and devices for closing and/or sealing punctures in tissue. In one illustrative embodiment, a device is provided for delivering and deploying an anchor, plug, suture, and a locking element adjacent to the opening in the vessel wall and/or tissue tract. In some cases, the plug may be configured to compress against the anchor when deployed in the tissue tract and/or opening in the vessel wall. In some cases, the suture may be automatically released from the device when the plug is compressed. In some cases, the device may include a motor to facilitate controlled, automatic compression of the plug.

### BRIEF DESCRIPTION OF THE DRAWINGS

The disclosure may be more completely understood in consideration of the following detailed description of various embodiments of the invention in connection with the accompanying drawings, in which:

FIG. 1-10 are perspective views and partial cut-away perspective views showing an illustrative procedure for sealing and/or closing a puncture in a vessel wall and/or adjacent tissue tract.

FIGS. 11-19 are partial cut-away schematic diagrams of an illustrative implantation device that may be used in the procedure of FIGS. 1-10.

FIG. 20 is a side-view drawing of a sheath having at least one perforation along its distal portion.

FIG. 21 is a side-view drawing of a sheath having at least one score line along its distal portion.

FIG. 22 is a side-view drawing of a sheath having a slit of variable width along its length.

FIGS. 23a and 23b are a side-view drawing of a sheath having a slit of variable width along its length, with the slit being open and shut, respectively.

FIGS. 24a and 24b are perspective drawings of a sheath having at least one weakened region, in the radially unexpanded and radially expanded states, respectively.

FIGS. 25a and 25b show side-views of a sheath formed in a folded manner, in the radially unexpanded and radially expanded states, respectively.

FIGS. 26a and 26b show end-on views of the sheath of FIGS. 25a and 25b.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

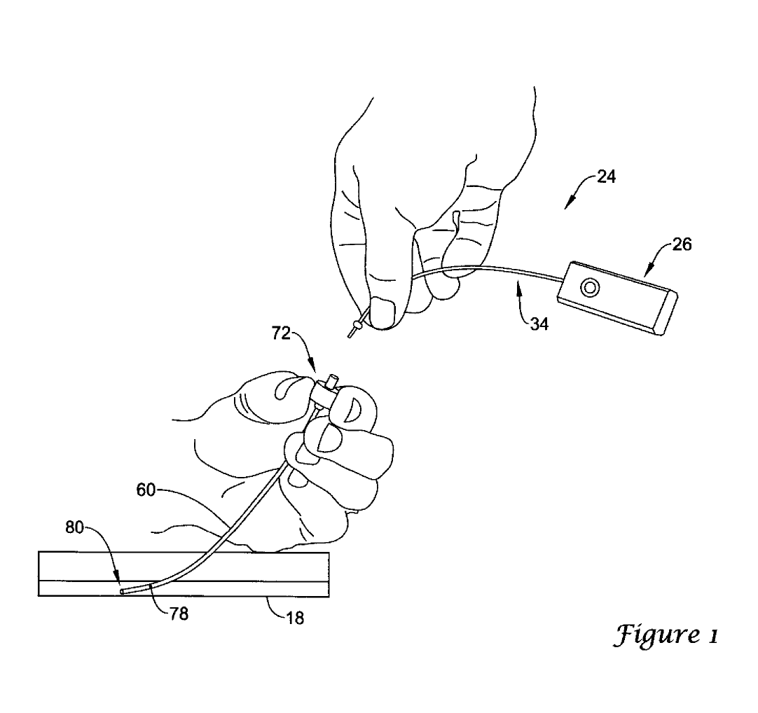

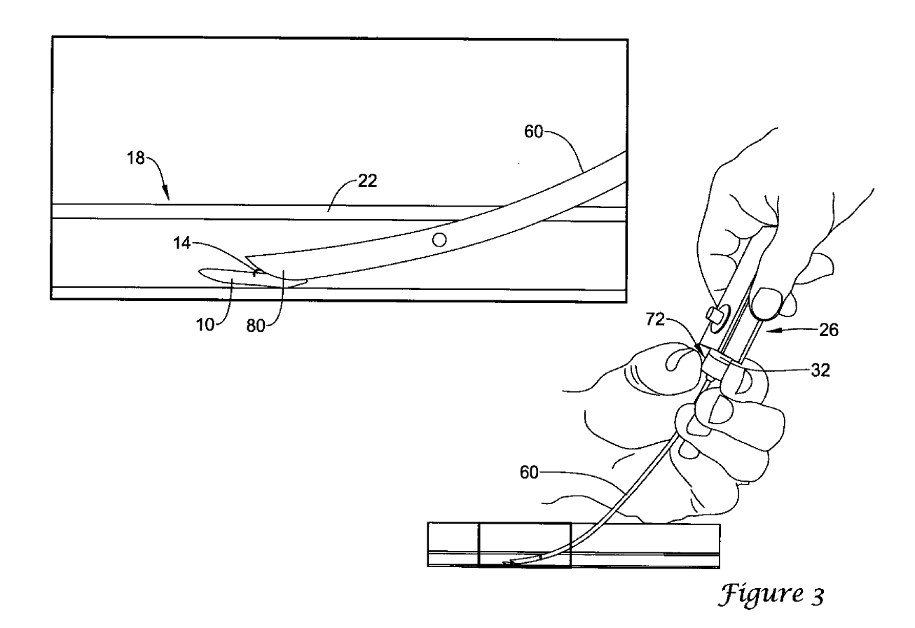

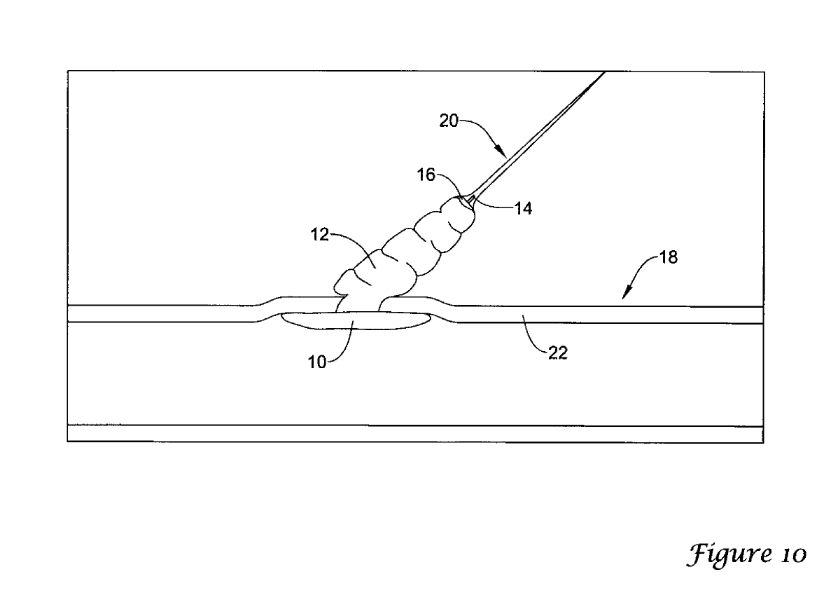

FIGS. 1-10 are perspective views and partial cut-away perspective views showing an illustrative procedure for sealing and/or closing a puncture in a vessel wall 22 and/or adjacent tissue tract 20 using an illustrative implantation device 24. In some cases, a medical procedure can be performed with a procedural sheath, which in some cases, may be different than the insertion sheath 60 described herein. In some cases, a guidewire (not shown) may be used to facilitate insertion of the insertion sheath 60 and/or the implantation device 24. A mating dilator (not shown) may be provided to support the insertion sheath 60 during advancement over the guidewire. In some cases, the vessel may be occluded by depressing the skin to temporarily stop the flow of blood therethrough.

A suitable bleed path position indicator may be used to correctly position a distal end of the insertion sheath 60 in the blood vessel 18. In some embodiments, at least one opening 78 in the insertion sheath 60 may define an entrance to a bleed path that may be used to determine if the distal end of the insertion sheath 60 is located within the lumen of the blood vessel 18. In some cases, the opening(s) 78 may be aligned with and fluidly communicate with a dilator (not shown). Blood may enter the bleed path through the distal opening(s) 78 and move proximally within the dilator (not shown). Blood moving through the bleed path may leak from a proximal bleed hole (not shown) in the dilator, thereby indicating the distal end of the insertion sheath 60 is disposed within the blood vessel 18. Once the proper position is located, the dilator (not shown), if used, may be removed and a device sheath 34 may be inserted into the insertion sheath 60, as shown in FIG. 1. However, other suitable position indicators and/or locators may be used, such as, for example, one or more bent wires, one or more interlocking buttons, one or more folded components, an inflatable balloon, a radially expanding disc, as well as other suitable position indicator and/or locator or combination thereof, as desired.

In some embodiments, the insertion sheath 60 may be maintained in the located position during and/or after removal of the dilator (not shown) or other position indicator. In some cases, an annular shaped locking ring or other suitable locking ring, such as an elastomeric o-ring, can be used to maintain the position of the insertion sheath 60. In other cases, a physician or medical technician may hold the insertion sheath 60 to maintain the position. In some embodiments, an indicator or other visual mark can be provided to verify that the proper location is maintained. In some cases, the insertion sheath 60 may include an orientation indicator (not shown) on a proximal end thereof to help orient the insertion sheath 60. In some cases, the orientation indicator may be a line, mark, shape, other indicator, or combination thereof, to aid a user in orienting the insertion sheath 60 relative to its position in the vessel. The implantation device 24 can then be inserted into the proximal end of the insertion sheath 60, as shown in FIG. 1. In some embodiments, insertion sheath 60 can include a hemostatic valve disposed therein. The implantation device 24 can be inserted through the hemostatic valve and connected to the insertion sheath 60. At the same time, an anchor 10 can be introduced into the blood vessel 18.

Implantation device 24 may include an anchor 10, a plug 12, a suture 14, and a locking element 16 for closing and/or sealing an opening in a blood vessel 18 and/or adjacent tissue tract 20 that was created to gain access to the blood vessel 18 to perform a medical procedure. The anchor 10 may be configured to engage an interior surface of the vessel wall 22. In some cases, the anchor 10 may be configured to partially or completely occlude the opening in the vessel wall 22, as desired. The anchor 10 may include a biodegradable material so that, over time, the anchor 10 is degraded, eroded, and/or absorbed in the body. In some cases, the anchor 10 may include a PLGA, PLLA, PGA or other degradable or erodable polymers, such as polyesters, polysaccharides, polyanhydrides, polycaprolactone, and various combinations thereof. In some cases, the anchor 10 may include a combination of the previously mentioned materials to impart a variable strength and/or degradation time profile in the anchor 10. One example anchor 10 that is configured to rapidly absorb and/or degrade is disclosed in Application Ser. No. 61/031,456, filed Feb. 26, 2008, which is hereby incorporated by reference. In some cases, the However, it is contemplated that other suitable anchors 10 may be used, as desired.

Suture 14 may include a proximal end, a distal end, and a length extending therebetween. The distal end of the suture 14 may be coupled to the anchor 10 with the suture 14 extending proximally therefrom. In some cases, the anchor 10 may include a raised portion including an eyelet to facilitate attachment of the distal end of the suture 14 to the anchor.

The suture 14 may include a biodegradable material so that, over time, the suture 14 is degraded, eroded, and/or absorbed in the body. In some cases, the suture 14 may include a PLGA, PLLA, PGA or other degradable or erodable polymers, such as polyesters, polysaccharides, polyanhydrides, polycaprolactone, and various combinations thereof.

In the illustrative embodiment, the plug 12 can be disposed about at least a portion of the suture 14 adjacent to the anchor 10 in the tissue tract 20 and/or opening of the vessel wall 22. The plug 12 may be configured to fill the space in the tissue tract 20 adjacent to the blood vessel 18 and/or the opening in the vessel wall 22 to close and/or seal the blood vessel 18 opening and/or tissue tract 20. In some examples, the plug 12 may include a material that swells to fill space in the tissue tract 20 and/or vessel wall 22 opening, such as by elastic expansion, fluid absorption, chemical reaction, as well as other suitable swelling and/or expansion. The plug 12 can be configured to promote hemostasis and/or clotting adjacent to the blood vessel 18. In one example, the plug may include collagen foam, gelatin foam, PEG or other hydrogel, starch powder, other suitable hemostatic materials, other suitable clot-promoting materials, as well as other suitable materials, as desired. In some cases, other materials can be used to provide control of thrombogenicity or hydration.

In the illustrative embodiment, the plug 12 may be generally cylindrical in shape with a lumen extending therethrough. As illustrated, the plug 12 is shown in an axially compressed state after it has been deployed in the tissue tract 20. In some cases, the plug 12 can be radially compressed prior to delivery, as desired.

The plug 12 may include a biodegradable material so that, over time, the plug 12 is degraded, eroded, and/or absorbed in the body. In one example, the plug 12 can include an elongated member formed from collagen foam or gelatin foam, such as, for example, GELFOAM® (Pharmacia & Upjohn, Inc., Bridgewater, N.J.) or Surgifoam™ (Johnson & Johnson, New Brunswick, N.J.). In some cases, the plug 12 can also include a hydrogel and/or a hemostatic material, if desired. Example hydrogels can include polyethylene glycols (PEG), including PEG 900, PEG 3350, and PEG 6000, as well as other suitable hydrogels, as desired. Examples of hemostatic materials can include starch powders, such as BleedArrest™ Clotting Powder (Hemostasis, LLC, St. Paul, Minn.). In one illustrative example, the starch powder can be disposed in or on the collagen or gelatin foam. In this illustrative example, the hydrogel can be coated on at least a portion of the collagen or gelatin foam and starch powder combination by, for example, drip coating, spray coating, or dip coating. However, other suitable methods of combining the collagen or gelatin foam, hydrogel, and starch powder can be used, as desired.

Some examples of plugs and plug materials that may be used in the closure device are disclosed in co-pending application Ser. No. 12/390,289, filed on Feb. 20, 2009, which is hereby incorporated by reference. In some cases, the plug 12 can include one or more voids, notches, slits, or other modifications to provide a desired axial compression of plug 12. Examples of plugs that may include voids, notches, slits, or other modification are disclosed in co-pending application Ser. No. 12/389,960, filed on Feb. 20, 2009, which is hereby incorporated by reference. In some cases, the illustrative plug 12 can be processed to have desired expansion characteristics. For example, the plug 12 can be tenderized to break down cell walls to increase the rate of expansion of the plug 12. Examples of plugs that have been tenderized or otherwise processed and methods of tenderizing or otherwise processing are disclosed in co-pending application Ser. No. 12/390,067, filed on Feb. 20, 2009, which is hereby incorporated by reference.

In the illustrative embodiment, one or more locking elements 16 can be used to help secure the plug 12 relative to the anchor 10. As illustrated, the locking element 16 can be disposed about at least a portion of the suture 14 proximal of the anchor 10 and the plug 12. The locking element 16 can be configured to slide over the suture 14 and compress the plug 12 during deployment. In some cases, the locking element 16 can be slid distally over the suture 14 to compress the plug 12, maintaining the distal portion of the suture 14 under tension. In some cases, the locking element 16 can be a knot, such as a compression knot that may exert a radial force on the suture 14. As such, the knot may have a friction force of 0.5 pounds, 1 pound, 1.5 pounds, 2.0 pounds, 2.5 pounds, 3.0 pounds, or other suitable friction force depending on the production of the knot. In any event, the friction force of the knot may be greater than the rebound force of the plug 12 to prevent the plug 12 from axially expanding after axial compression.

In an illustrative embodiment, the locking element 16 may be separate and independent from the suture 14. In some cases, the locking element 16 may include a filament that is independent of the suture 14. In some cases, the filament of the locking element 16 may have a larger radial diameter than the suture 14 so that the locking element 16 has a sufficient size to contact the proximal end of the plug 12 for axial compression without penetrating into the plug 12.

In other cases, the locking element 16 can be a sliding cinch, a disc shaped retainer, or other device. In some cases, the locking element 16 may be capable of sliding relative to the suture 14 upon an exertion of force. In other cases, the locking element 16 can be configured to slide in a distal direction relative to the suture 14, but not in a proximal direction. An example locking element is disclosed in co-pending application Ser. No. 12/389,847, filed on Feb. 20, 2009, which is hereby incorporated by reference.

The locking element 16 may include a biodegradable material so that, over time, the locking element 16 is degraded, eroded, and/or absorbed in the body. In some cases, the locking element 16 may include a PLGA, PLLA, PGA or other degradable or erodable polymers, such as polyesters, polysaccharides, polyanhydrides, polycaprolactone, and various combinations thereof.

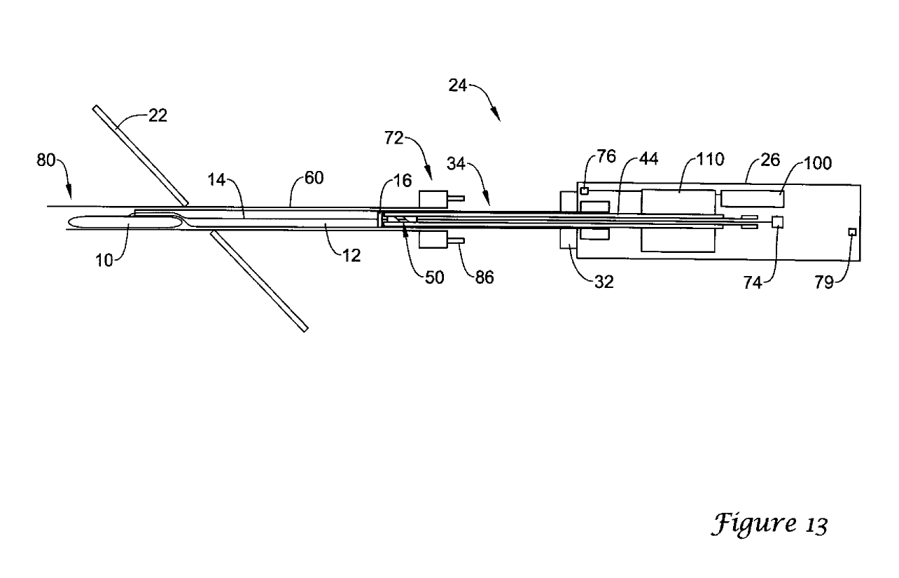

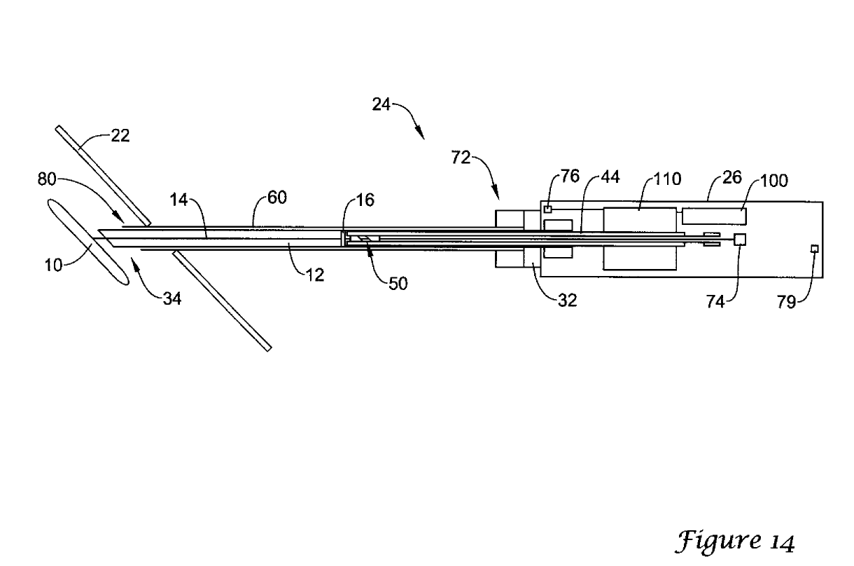

The implantation device 24 may include a device handle 26 and a device sheath 34. The device sheath 34 may be a tubular member having a proximal end fixedly coupled to the device handle 26. The anchor 10 can be disposed adjacent the distal end of the device sheath 34, either within the device sheath 34, partially within the device sheath 34, or outside the device sheath 34. The anchor 10, when positioned outside the device sheath 34 generally in axial alignment with the device sheath 34, may at least partially deform the distal end of the device sheath 34 about the anchor 10, as seen in FIG. 13, for example. This arrangement permits the distal end of the plug 12 to be maintained in close proximity to the anchor 10, thereby minimizing any gap between the two elements. As the anchor 10 is positioned generally transverse to the distal end of the sheath, the deformed portion of the distal end of the device sheath 34 may assume a shape or form substantially similar to a proximal portion of the device sheath 34, as seen in FIG. 14, for example. The plug 12, suture 14, and locking element 16 can be disposed within the device sheath 34.

The device handle 26 may include a control handle connector 32 configured to attach the implantation device 24 to an insertion sheath 60. The illustrative implantation device 24 may allow for ambidextrous use and provide controlled deployment of the anchor 10, plug, 12, suture 14, and/or locking element 16.

In some embodiments, a push rod 44 can be provided having a proximal end disposed in the device handle 26 and a distal end disposed in the device sheath 34. A proximal end of the push rod 44 can be operatively coupled to a motor 110 within the device handle 26, where the motor 110 is configured to adjust the relative relationship of the push rod 44 and device handle 26 in response to the activation of a force trigger 79. Motor 110 may be operatively connected to an energy source and/or control system 100 disposed within the device handle 26. Force trigger 79 may be operatively connected to control system 100 to provide an input thereto. The force trigger 79 may be activated by a predetermined tensile force applied when the anchor 10 is positioned against the vessel wall 22 of the blood vessel 18 and the device handle 26 is tensioned proximally away from the blood vessel 18. Alternatively, the force trigger 79 may be activated by an optional, manually-activated switch in communication with an exterior of the device handle 26, where the optional, manually-activated switch may be actuated by a user of the implantation device 24. The optional, manually-activated switch is shown in the Figures, but need not be present in all embodiments. For example, embodiments utilizing an automatic or tensile force-activated force trigger may not have a separate manually-activated switch. However, it is contemplated that some embodiments may have an automatic force trigger and a manually-activated switch.

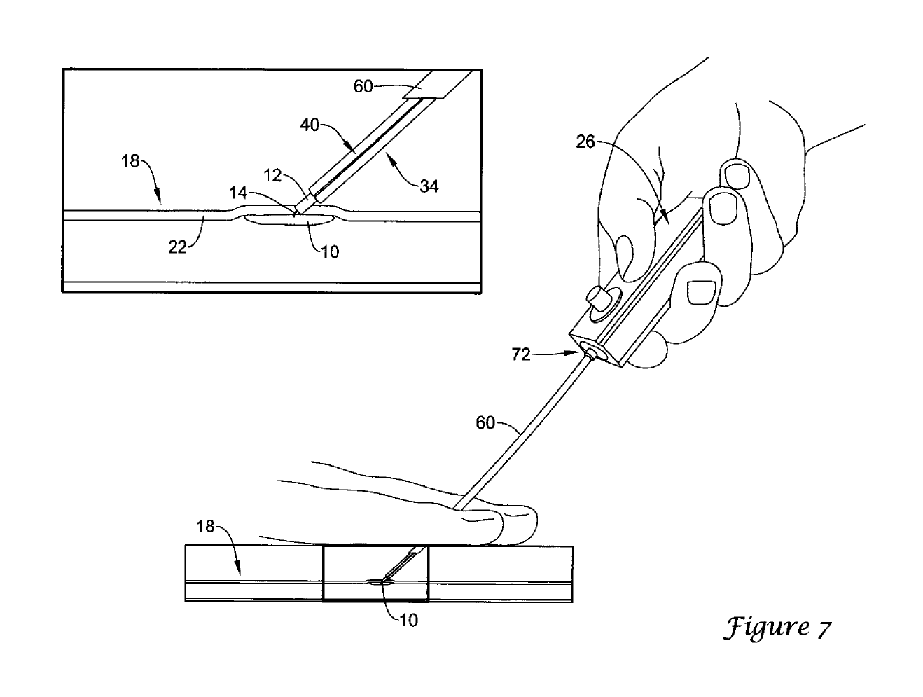

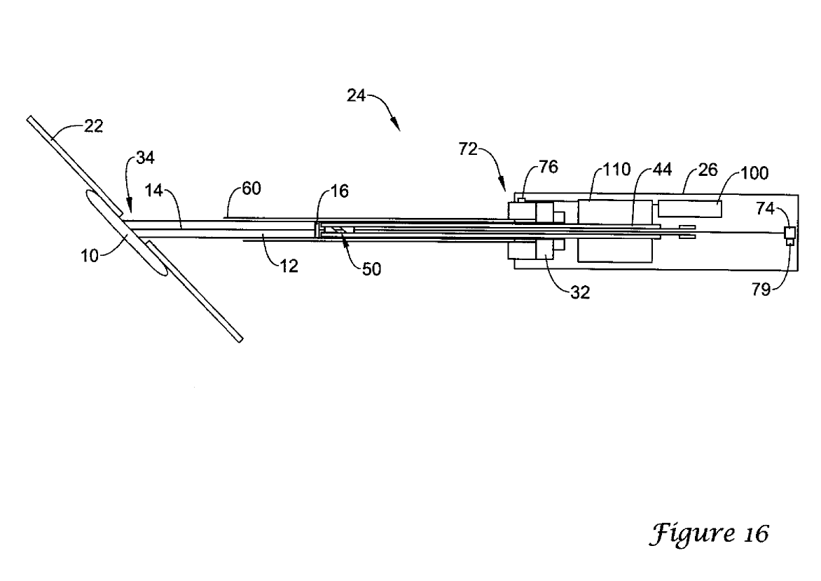

Activation of force trigger 79 may cause control handle connector 32, hub 72, and insertion sheath 60 to be withdrawn proximally relative to the anchor 10 to create a small gap for deployment of plug 12, as seen in FIGS. 7 and 16, for example. Withdrawal may be facilitated manually, by actuation of the motor 110, a spring mechanism, or other operatively connected means as appropriate. Device sheath 34 and the device handle 26 remain fixed in position relative to the anchor 10. Following withdrawal of insertion sheath 60, motor 110 may be actuated manually, sequentially, or automatically to extend push rod 44 distally to deploy the plug 12.

Motor 110 may provide a controlled displacement and compression speed for deployment of the plug 12. A motor-driven system can reduce the forces applied by a user to the implantation device 24 during deployment, may provide gradual acceleration and deceleration of the compression movements within the device, and may reduce peak actuation forces to avoid damage to the plug 12 during compression or to avoid fracturing the anchor 10.

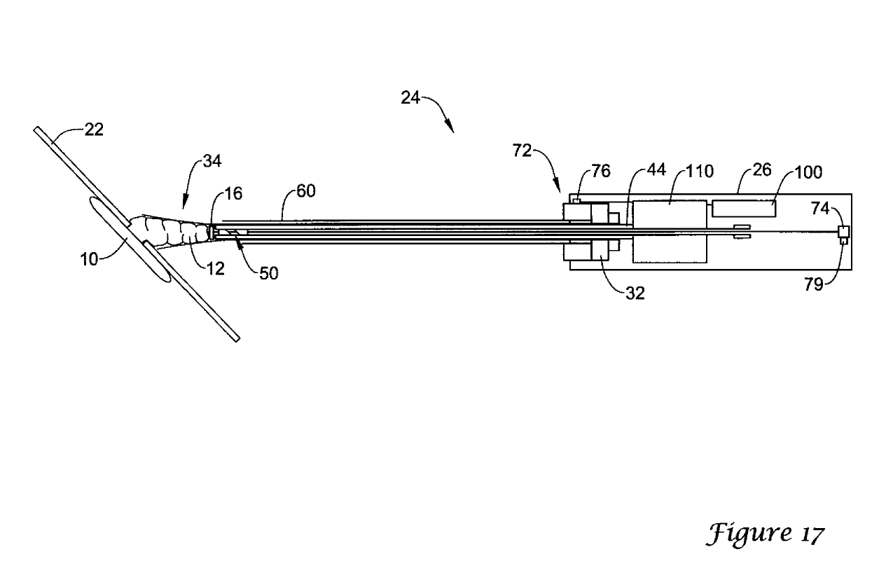

Push rod 44 may advance against the locking element 16 to compress the plug 12, as shown in FIG. 17. The plug 12 may be compressed and secured in the compressed state by the locking element 16. In one example, the locking element 16 may have a compressive force on the suture creating a friction force in the locking element of 0.5 pounds, 1 pound, 1.5 pounds, 2 pounds, or other suitable friction force, as desired. Accordingly, the force exerted by the push rod 44 onto the locking element 16 may be greater than the friction force between the locking element 16 and the suture 14. Further, the plug 12 may exert a rebounding force on the locking element 16 trying to return to the non-axially compressed position. However, the friction force of the locking element 16 may be configured to be greater than the rebounding force of the plug 12.

In some embodiments, the push rod 44 can include a suture release member 50 such as a collet, but other suitable push rods may be used, as desired. The distal end of the push rod 44 can include a collet lock ring that is configured to have a releasable engagement with the suture 14. In some cases, the distal end of the push rod 44 can be coupled to the suture 14. A release bead can be disposed about a portion of the push rod 44 a distance from the collet lock ring. The release bead may slide relative to the push rod 44 and is configured to engage the collet lock ring and slide the collet lock ring off of the distal end of the push rod 44, releasing the suture 14.

A proximal tubular member can be disposed about at least a portion of the push rod 44 proximal of the release bead. A distal tubular member can be disposed about the push rod 44 and have a proximal end configured to engage the release bead and a distal end configured to engage locking element 16. The distal tubular member may be configured to slide over the collet lock ring. In some cases, the release bead may simultaneously or concurrently pass over the push rod 44 and engage the collet lock ring to automatically release the suture 14 from the implantation device 24. The push rod 44, proximal tubular member, release bead, distal tubular member, and collet lock ring may be disposed within the device sheath 34.

In some embodiments, the push rod 44 and/or the proximal tubular member and the distal tubular member may be a coil having a number of turns. However, it is contemplated that a suitable tubular member having a sufficient pushability and flexibility may be used, as desired.

In some embodiments, the suture 14 may include an attachment element such as a loop, disposed at the proximal end of the suture 14. The suture 14 may be released from the device handle 26 by releasing the attachment element. The collet described herein may also be considered an attachment element, as releasing either element may result in the suture 14 being released from the device handle 26.

In some embodiments, the suture release member 50 may include a cutting element configured to cut the suture 14 within the device sheath 34 following deployment of the plug 12 to release the suture 14 from the device handle 26.

The implantation device 24 may also include a control handle connector 32 configured to engage a hub 72 of the insertion sheath 60. The control handle connector 32 can be configured to be housed in the distal end of the device handle 26 or extend partially out of the distal end of the device handle 26. The control handle connector 32 may include a lumen configured to receive a proximal region of the device sheath 34 such that the device sheath 34 may pass through the control handle connector 32.

Insertion sheath 60 may include a hub 72 configured to couple the insertion sheath 60 to the control handle connector 32. The hub 72 may include one or more pins and/or protrusions 86 that are configured to engage the control handle connector 32 to mate the insertion sheath 60 to the implantation device 24. In some embodiments, the control handle connector 32 of the device handle 26 may mate with the hub 72 in only one orientation. In some embodiments, the hub 72 may include a major radial axis that is offset from the major radial axis of the device handle 26. In some cases, the distal end 80 of the insertion sheath 60 may be beveled to accommodate the anchor 10 at the desired deployment angle for proper approximation to the vessel wall 22 of the blood vessel 18. In some embodiments, the implantation device 24 can be inserted into the insertion sheath 60 at an orientation offset from the insertion sheath 60, but this is not required. It is contemplated that other suitable connectors may be used instead of the illustrative control handle connector 32 and hub 72, as desired.

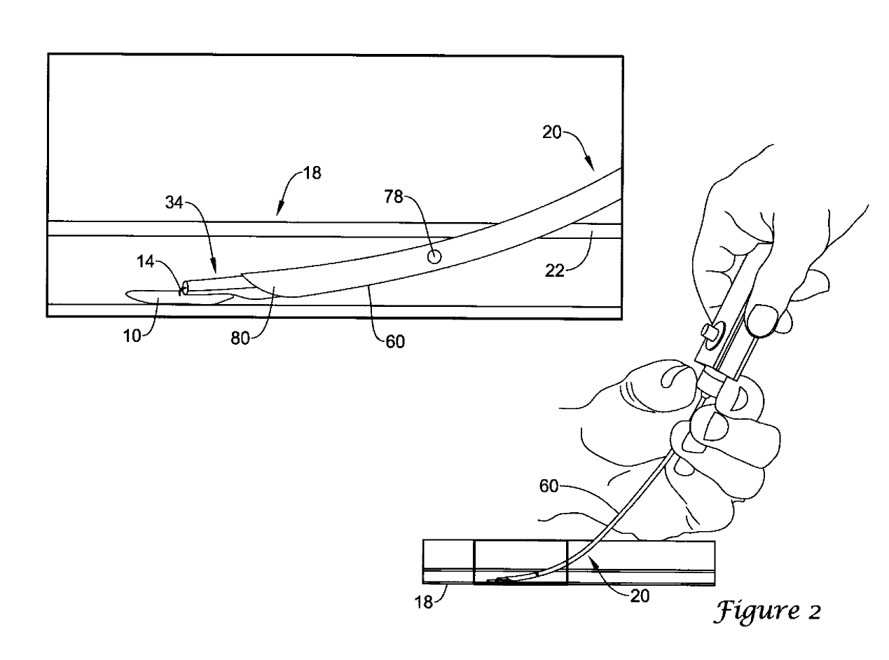

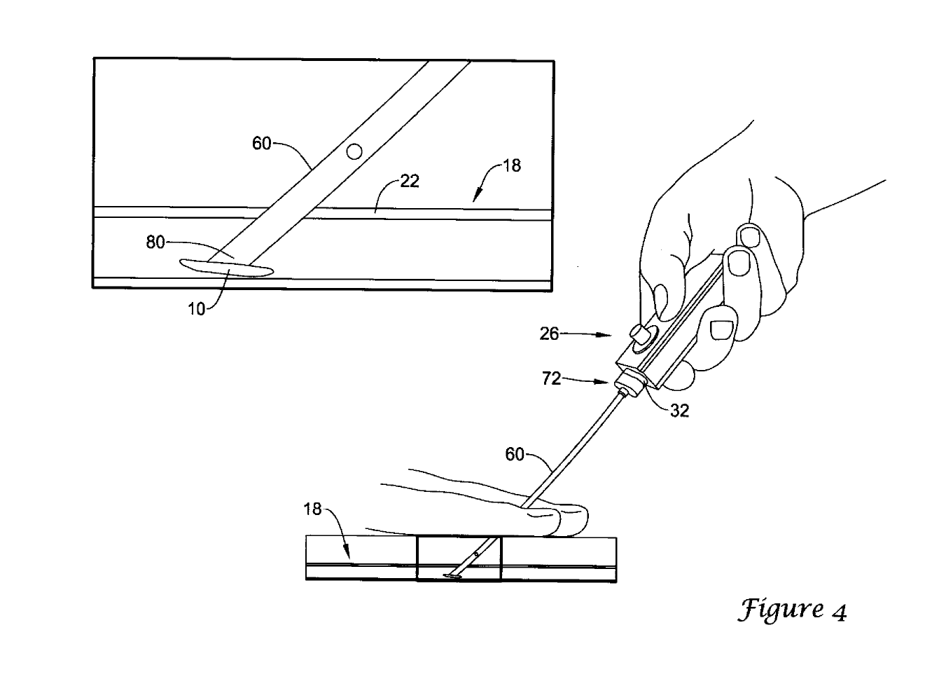

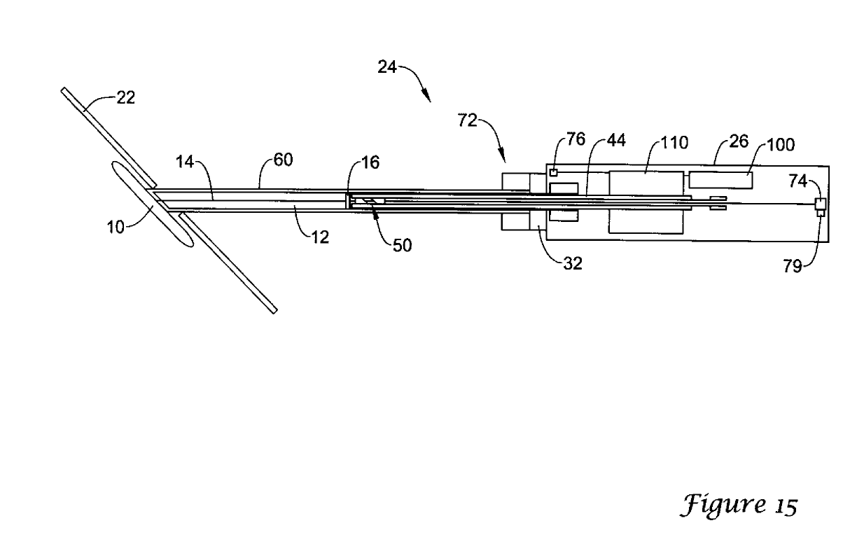

In some embodiments, the device sheath 34 of the implantation device 24 may be completely inserted into the insertion sheath 60. As shown in FIGS. 2 and 14, when the implantation device 24 is completely inserted, the anchor 10 is deployed out the distal end of the insertion sheath 60 into the blood vessel 18. When deployed, the anchor 10 may be initially spaced from the beveled distal end 80 of the insertion sheath 60, but, as shown in FIGS. 4 and 15, can be subsequently retracted, in some cases automatically, against the beveled distal end 80.

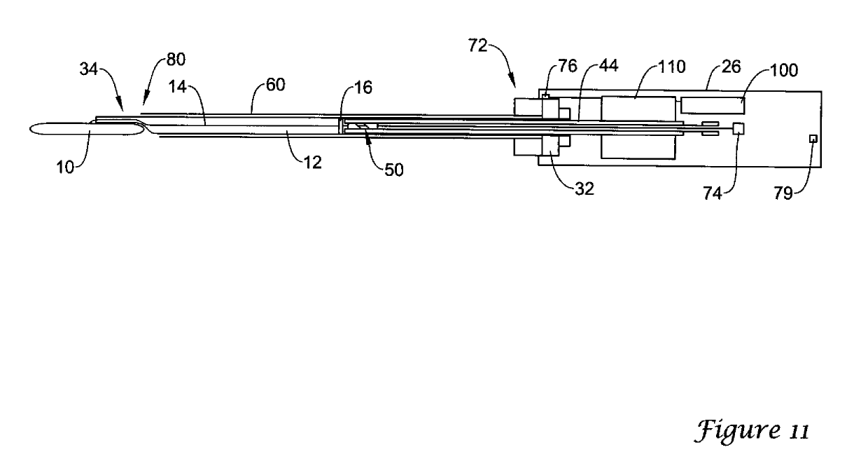

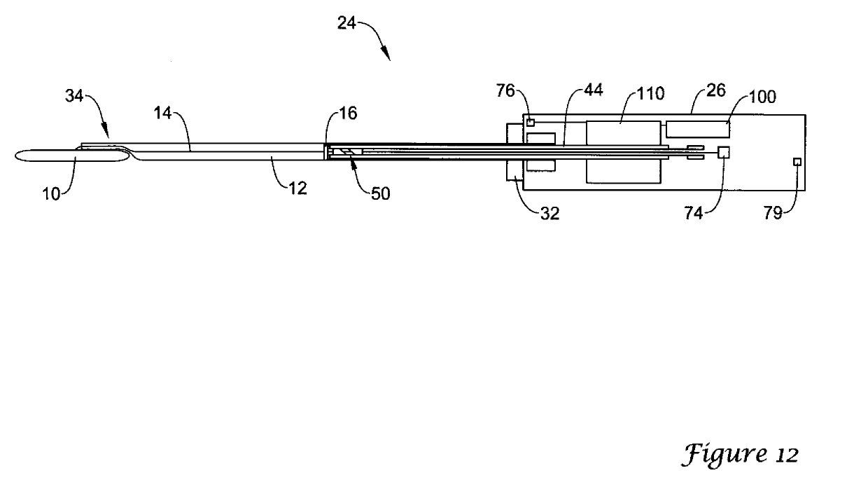

FIG. 11, for example, shows the implantation device 24 secured to the insertion sheath 60 before the anchor 10 has been seated. To do this, in some embodiments, the device handle 26 of the implantation device 24 can be rotated relative to the insertion sheath 60 to align the hub 72 with the control handle connector 32. In some embodiments, the implantation device 24 can be rotated about 90 degrees when viewed from the proximal or distal end. The rotation may lock the control handle connector 32 to the hub 72 and actuate a seating mechanism 74. In some embodiments, the hub 72 may engage the control handle connector 32 to activate a position trigger 76. Position trigger 76 may be operatively connected to seating mechanism 74 such that activation of position trigger 76 may automatically actuate the seating mechanism 74. However, it is contemplated that other attachment, alignment, and/or release mechanisms may be used to connect the insertion sheath 60 to the implantation device 24 and to seat the anchor 10 against the distal end of the insertion sheath 60, as desired. Examples of such components that may be used can include interlocking snaps, torsion springs, spring releases, keys, push pins, and other suitable components, as desired.

In some embodiments, the seating mechanism 74 may translate the implantation device 24, including device handle 26, anchor 10, suture 14, and device sheath 34, proximally relative to the insertion sheath 60 while the insertion sheath 60 is held in a fixed position. In some embodiments, the seating mechanism 74 may translate the insertion sheath 60 distally relative to the implantation device 24 while the implantation device 24 is held in a fixed position. In some embodiments, a spring mechanism or motor may be the seating mechanism 74 that provides the translation discussed herein. In other embodiments, the seating mechanism 74 may be a spring mechanism or a motor configured to apply tension to the suture 14 by axially moving the proximal end of the suture 14 proximally within the device handle 26. The relative movements described herein may translate the anchor 10 into contact with, or into close proximity to, the beveled distal end 80 of the insertion sheath 60. Alternatively, the seating mechanism 74 may include a combination of the above-described elements and/or motions which provide the relative movement required. The relative positioning of the anchor 10 and the distal end 80 after the anchor 10 has been seated as discussed herein may be seen, for example, in FIGS. 4 and 15.

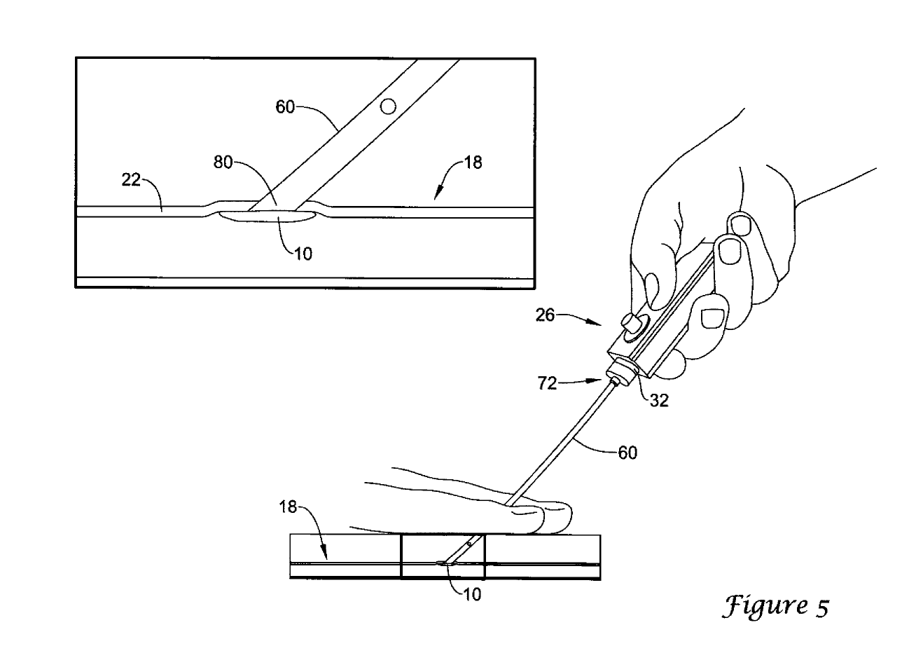

As shown in FIG. 5, the device handle 26 and insertion sheath 60 can then be retracted or translated proximally to seat the anchor 10 against the interior surface of the vessel wall 22. With the anchor 10 seated against the interior surface of the vessel wall 22, tension may be continually applied to the device handle 26 of implantation device 24.

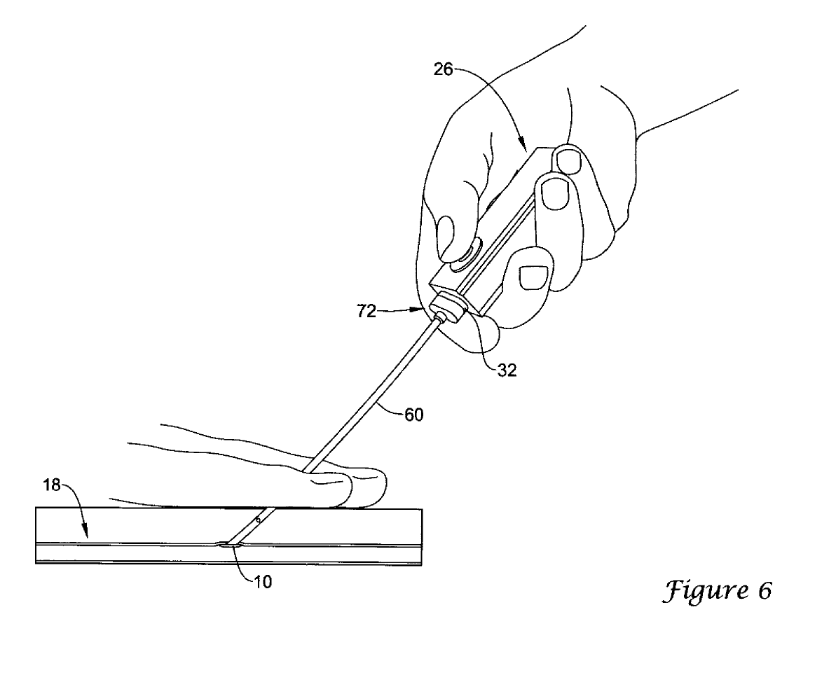

FIGS. 5 and 6 show the anchor 10 seated against the interior of the vessel wall 22 or arteriotomy. The suture 14 is coupled to the anchor 10 and extends proximally through the tissue tract 20. The plug 12 is disposed over the suture 14 adjacent the anchor 10 and the locking element 16 is disposed about the suture 14 proximal of the plug 12. The plug 12, suture 14, and locking element 16 may be disposed, at least partially, within the device sheath 34. At the step shown in FIG. 6, force trigger 79 may be activated automatically by tension on suture 14 as implantation device 24 is pulled proximally, or force trigger 79 may optionally be activated by the user pressing a manually-activated switch, as illustrated in FIG. 6.

As shown in FIGS. 7 and 16, the insertion sheath 60 may be retracted a distance from the anchor 10 and/or opening in the vessel wall 22 to provide a gap or an area for plug deployment. Insertion sheath 60, hub 72, and control handle connector 32 may be retracted proximally such that the hub 72 and the control handle connector 32 are at least partially withdrawn into the device handle 26. Control handle connector 32 and the hub 72 may be completely withdrawn into the device handle 26 in some embodiments. In one illustrative example, the distance may be about two-thirds of the length of the plug 12. For example, if the plug 12 is about three-quarters of an inch long in a non-axially compressed state, the distance that the insertion sheath 60 can be retracted may be about one-half inch. However, it is contemplated that others suitable distances may be used, as desired. As discussed above, the device sheath 34 and the device handle 26 remain fixed in position relative to the anchor 10 and blood vessel 18.

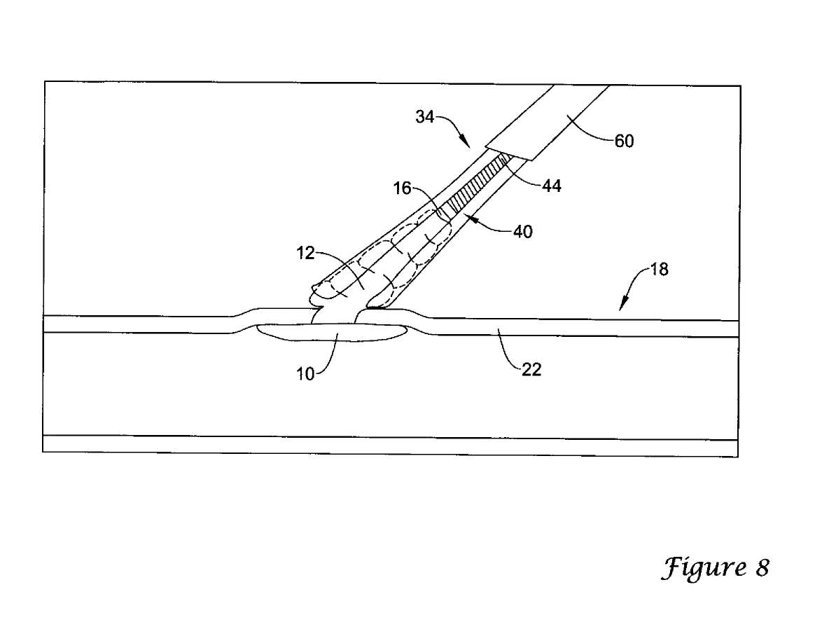

As shown in FIGS. 8 and 17, the plug 12 may be deployed in the tissue tract 20 while continuing to apply tension to the implantation device 24. The distal end of device sheath 34 may include at least one longitudinal slit 40. Deployment of the plug 12 may include axial compression and/or radial expansion. The at least one longitudinal slit 40 may widen as the plug 12 is axially compressed and/or radially expanded. The distal end of the device sheath 34 provides reduced friction for distal movement of the plug 12 during deployment compared to the plug 12 directly contacting the tissue tract 20. This may result in reduced distal deployment force, improved plug cohesion, and/or reduced trauma to the tissue tract 20 and/or blood vessel 18.

Note that in some cases, the openings at or near the distal end of the device sheath may include a material that is capable of softening and/or dissolving when exposed to selected conditions.

With continued tension to the implantation device 24, an automatic suture release member 50 may be actuated to release the suture 14 from the implantation device 24. The automatic suture release member 50 may include other appropriate means of releasing the suture 14, such as, but not limited to, a collet that may release a proximal end of the suture 14, a loop disposed at a proximal end of the suture 14 which may be released from the device handle 26, or a cutting element that may cut the suture 14. Suture 14 may be released or cut within or external to tissue tract 20, within or external to the insertion sheath 60, within or external to the device sheath 34, or a combination thereof. For example, suture 14 may be cut within the insertion sheath 60 while the insertion sheath 60 is disposed within the tissue tract 20.

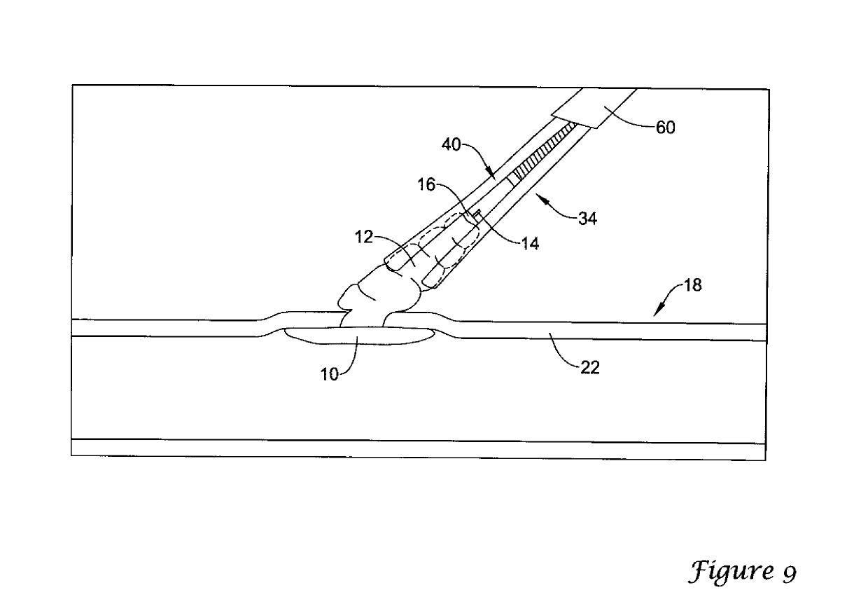

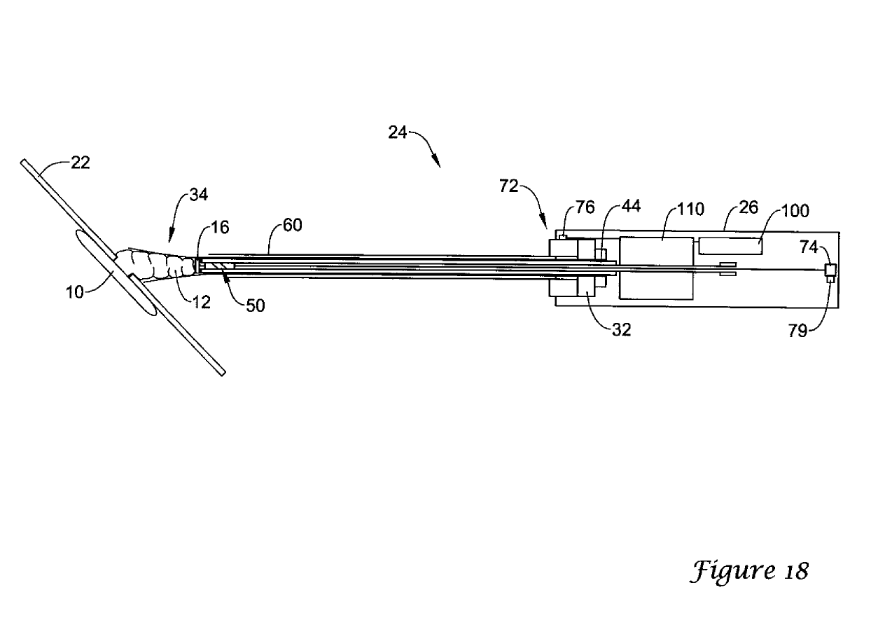

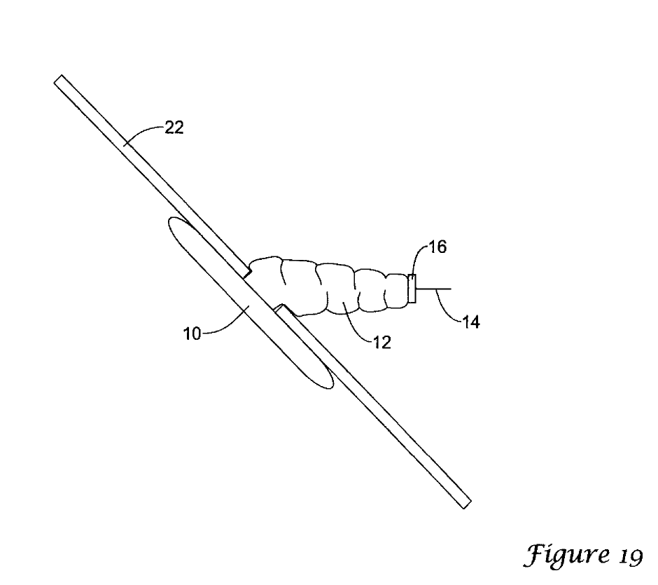

As shown in FIGS. 9-10 and 18-19, the suture 14 is released from the implantation device 24 and then, the insertion sheath 60 and the implantation device 24 can be removed from the tissue tract 20 leaving the anchor 10, plug 12, suture 14, and locking element 16 to seal and/or close the opening in the vessel wall 22 and/or tissue tract 20. If the suture 14 extends proximally of the locking element 16 outside of the tissue tract 20, the extra length can be removed, such as, for example, by cutting. In other cases, the suture 14 may have a length such that no extra cutting may be needed. When the plug is exposed to a fluid, such as blood for example, the plug 12 can expand to fill the tissue tract 20 and/or opening in the vessel wall 22.

While the foregoing has described the implantation device 24 in detail, this is not meant to be limiting in any manner. It is contemplated that a suitable apparatus for sealing and/or closing an opening in a vessel wall and/or tissue tract can include other combinations of the above-described features.

Many of the steps described herein may occur automatically to streamline the procedure from a user perspective. For example, after inserting the device sheath 34 into the insertion sheath 60, the motor-driven deployment mechanism can be actuated to snug the anchor 10 against the end of the insertion sheath 60, translate the anchor 10 against the vessel wall 22, retract the insertion sheath 60 to provide clearance for deployment of the plug 12, compress and deploy the plug 12, cinch or lock the implant components to the vessel wall 22, and trim the suture 13 to length, all in sequence automatically.

Alternatively, various steps may be performed or actuated by the user when the user desires the particular steps to be performed. Various combinations of automatic, triggered, and manual steps can be utilized in different embodiments, with one or more motors providing plug compression and deployment or other required motions. For example, translating the anchor 10 against the vessel wall 22 may be done manually, while other steps are performed automatically.

As seen in FIGS. 8-10 and 17-19, the plug 12 may expand radially when forced longitudinally against the anchor 10. Such a radial expansion of the plug 12 may be accommodated more easily if the distal end of the device sheath 34 is allowed to expand fully or partially along with the expansion of the plug 12. For instance, allowing a distal portion of the device sheath 34 to expand may help prevent uncontrolled displacement of the plug 12 or damage to the plug 12, which may in turn help prevent bleeding complications and/or failure of the vascular closure device system. In particular, it may be especially desirable that the device sheath 34 can protect the plug 12 during introduction and deployment, can expand to accommodate the shape change of the plug 12 during deployment without damaging the plug 12, and can collapse or be folded up for recapture after deployment of the plug 12.

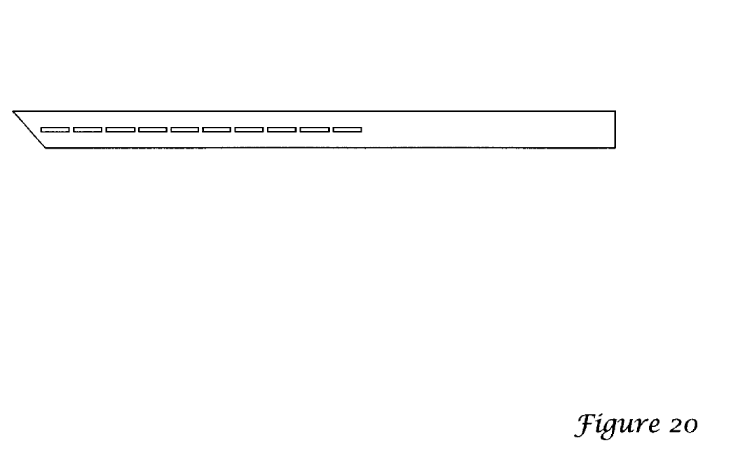

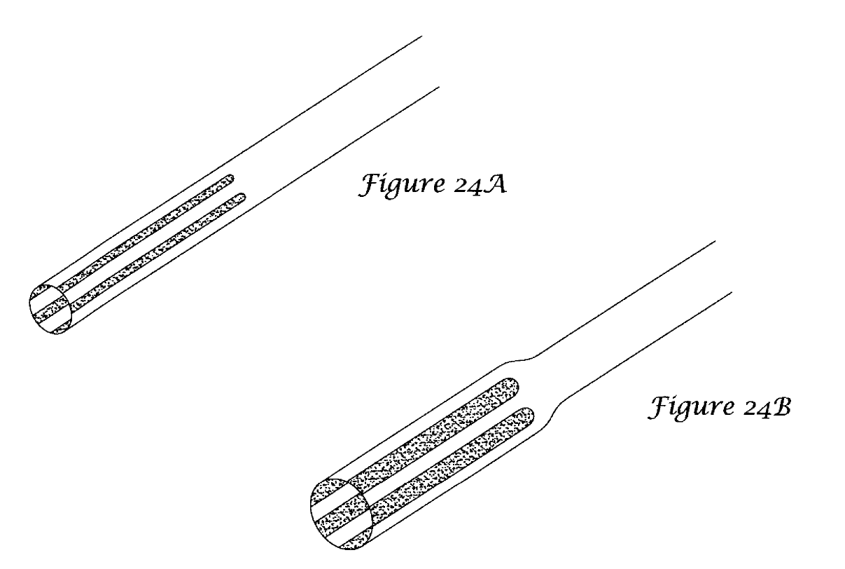

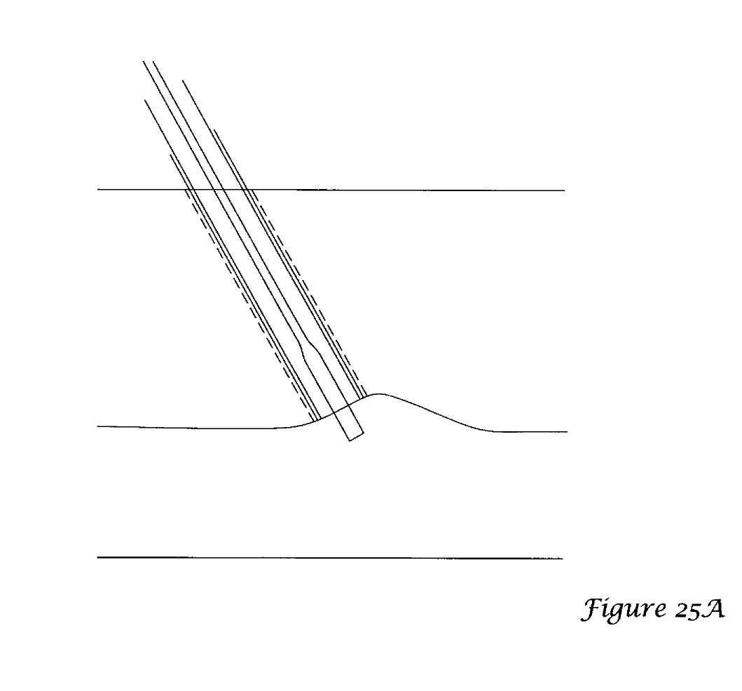

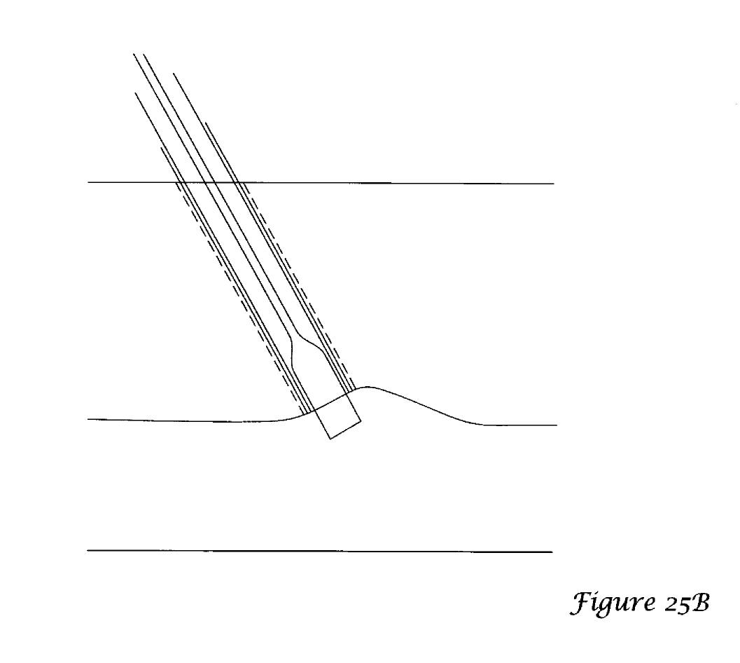

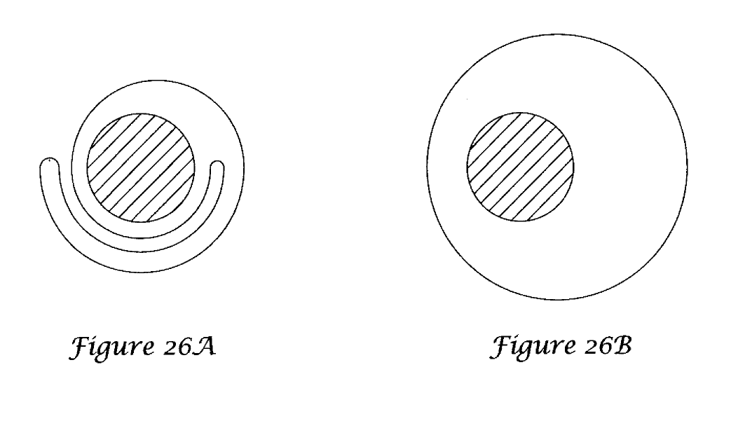

FIGS. 20-26 and the discussion that follows are directed toward various mechanisms that allow the distal end and/or the distal portion of a device sheath 34 to expand radially as needed, which may provide some or all of the above advantages. More specifically, FIGS. 20-23 are directed toward ways of weakening part of the distal portion of a device sheath 134 by including opening or score lines therein, so that it may tear in a predetermined manner. The opening may be held closed by a weak adhesive tacking which releases as the plug is deployed. FIGS. 24a and 24b are directed toward ways of embedding weakened portions into the distal portion of a device sheath 234 so that it may deform in a predetermined manner. FIGS. 25a, 25b, 26a and 26b are directed toward a way of folding a device sheath 334 so that it unfolds to expand in a predetermined manner. Each of these device sheaths may have a relatively small introduction profile, may have a distal portion that can expand to a larger diameter than the introduction profile during use, and can be collapsed again to a smaller profile for removal.

FIG. 20 is a side-view drawing of a device sheath 334 having at least one perforation 161 along its distal portion. The device sheath 134 can be used in place of device sheath 34 described above. The at least one perforation 161 itself may be formed as a linear series of apertures through or weaknesses in the wall of device sheath 134. In general, any suitable wall structure may be used, so that as the plug 12 expands radially within or near the distal end of device sheath 134, the perforations are easily torn. Note that the at least one perforation 161 ensures that in the vicinity of the plug 12 the distal portion of device sheath 134 expands radially to the size of the plug 12. Proximal to the plug 12, the at least one perforation 161 may not separate, leaving the device sheath 134 at an unexpanded size.

In some cases, the length of the at least one perforation 161 is approximately equal to the length of the plug 12 before deployment. In other cases, the at least one perforation 161 is shorter or longer than the length of the plug before deployment.

Although the at least one perforation 161 is shown in FIG. 20 as extending longitudinally along device sheath 134, there may be a tangential component to the perforation. For instance, the at least one perforation 161 may be helical in shape, and may precess around a longitudinal axis of device sheath 134. As another example, the perforation may include “wiggles”, or generally lateral oscillations about a generally longitudinal path. As a further example, the perforation may include irregularities in its path, optionally along with a helical shape and/or lateral oscillations.

Although only one perforation 161 is shown in FIG. 20, there may be two, three, four, five, six, seven, eight or more than eight perforations 161. In general, each at least one perforation 161 extends proximally from the distal end of device sheath 134 or from a point near the distal end of device sheath 134. In some cases, the perforations are distributed evenly around a circumference of device sheath 134. In other cases, the perforations are distributed unevenly around the circumference of device sheath 134. In some cases, the perforations 161 are generally parallel to each other along device sheath 134, meaning that they remain generally equidistant from the adjacent perforations 161 along the length of the perforations 161. In other cases, the perforations 161 may get closer to or farther away from the adjacent perforations 161 along their lengths.

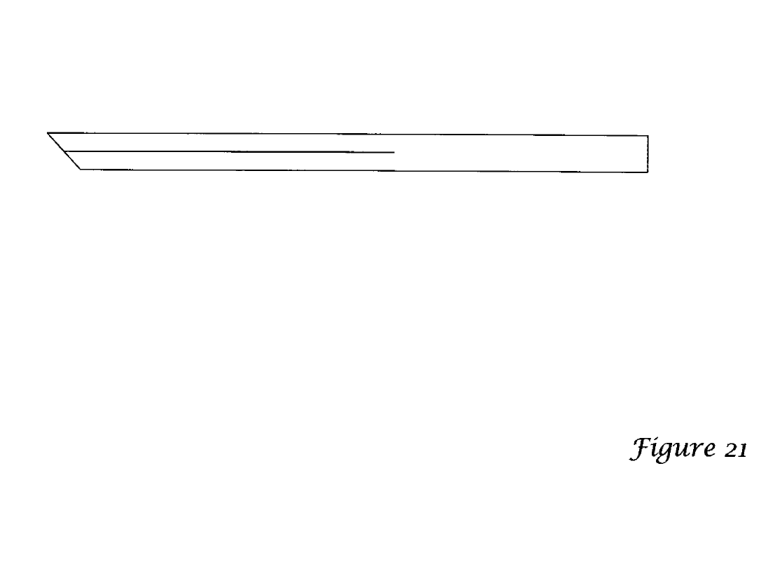

FIG. 21 is a side-view drawing of device sheath 134 having at least one score line 162 along its distal portion. The at least one score line 162 may be a generally thinned portion of the wall of device sheath 134, which generally does not go all the way through the wall of device sheath 134. As with the at least one perforation 161, the at least one score line 162 may provide a weakened portion in the wall of device sheath 134, along which the wall may tear when the plug 12 is expanded radially within device sheath 134.

Much like the at least one perforation 161, the at least one score line 162 may be present in numbers of one, two or more than two, may be purely longitudinal or may have a tangential component, may or may not be evenly spaced around the circumference of device sheath 134, and so forth. In some cases, device sheath 134 may include both at least one score line 162 and a at least one perforation 161, which may even be present along the same linear path on the sheath wall.

In addition to perforations 161 and score lines 162, the sheath may also include one or more slits that extend all the way through the sheath wall. Such a slit or slits may be present along the same linear path as a score line and/or a perforation along the sheath wall. In some cases, the slit may not extend all the way to the distal end of device sheath 134, but may stop proximal to the distal end. Such a configuration may be beneficial, in that device sheath 134 may provide stability during initial placement and positioning of the device, but not offer significant resistance to plug deployment.

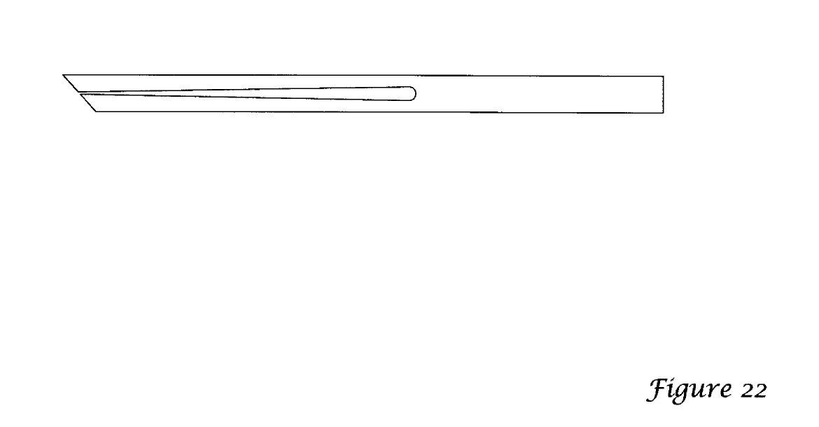

FIG. 22 is a side-view drawing of device sheath 134 having a slit 163 of variable width along its length. The slit 163 in FIG. 22 is shown as extending from the distal end of the sheath 160, and having an increasing width away from its distal end.

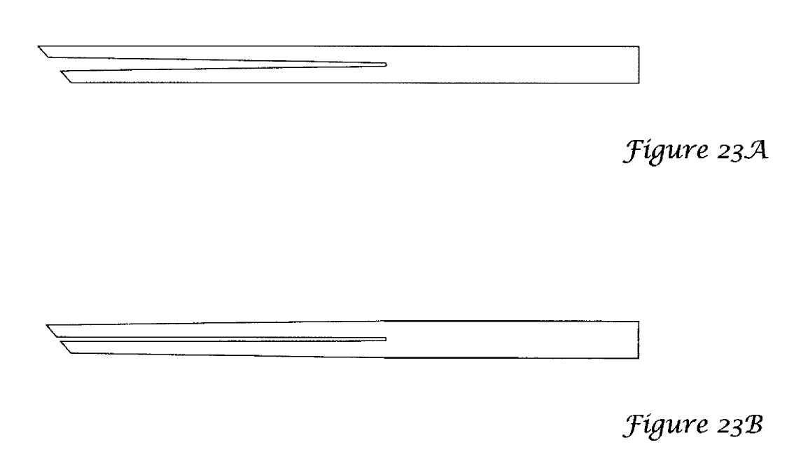

FIG. 23a is a side-view drawing of device sheath 134 having another slit 164 of variable width along its length. The slit 164 in FIG. 23a is shown as extending from the distal end of device sheath 134, and having a decreasing width away from its distal end. FIG. 23b is a side-view drawing of the same device sheath 134 as in FIG. 23a, but with slit 164 pressed shut. The device sheath 134 may be introduced with the shut configuration of FIG. 23b.

In some cases, device sheath 134 may have a combination of slits 163, 164 that have increasing and decreasing widths away from their respective distal ends.

The perforations 161, score lines 162 and slits 163, 164 of FIGS. 20-23 may all be used interchangeably to provide stability during initial placement and positioning, but not offer resistance to plug deployment. As device sheath 134 is retracted, the plug 12 remains placed against the anchor and artery and does not retract with the sheath 160, which may desirably lead to increased reliability. In general, device sheath 134 may be applicable to methods of shape-change deployment of an implanted device without damage to the implanted device, methods of arteriotomy closure, and methods of manufacture of a medical implant. The device sheath 134 may show decreased friction during deployment, and may therefore desirably allow use of a larger plug 12, which may in turn provide improved hemostasis. The device sheath 134 may improve the performance of anchor/plug/cinch devices by keeping the plug 12 against the anchor at all times, so that the plug 12 does not displace proximally during sheath retraction, and by reducing the amount of force required to deliver the plug.

FIGS. 20-23 are directed toward ways of weakening part of the distal portion of device sheath 134 by including openings or score lines therein, so that it may expand in a predetermined manner. FIGS. 24 is directed toward ways of embedding weakened portions into the distal portion of a device sheath 234 so that it may expand in a predetermined manner. Device sheath 234 may be used in place of device sheath 34.

FIGS. 24a and 24b are perspective drawings of device sheath 234 having at least one weakened region 261, in the radially unexpanded and radially expanded states, respectively. Sheath 260 can be used in place of insertion sheath 60 described above. As with the perforations 161, score lines 162 and slits 163 discussed above, the at least one weakened region 261 may be present in numbers of one, two or more than two, may be purely longitudinal or may have a tangential component, may or may not be evenly spaced around the circumference of device sheath 234, may extend to the distal end of device sheath 234, may extend distally to a point near the distal end of device sheath 234, may be used interchangeably with the perforations 161, score lines 162 and slits 163 discussed above, and so forth.

The at least one weakened region 261 may be regions in which the sheath wall is thinned, in which the sheath wall may be made from a more deformable material, and/or in which the sheath wall may be made from a harder material. In some cases, the at least one weakened region 261 may include reinforcement such as a fibers and/or wires, such as longitudinally oriented nitinol wires. Such wires may provide integrity to device sheath 234, and may circumferentially expand to accommodate a radially expanded plug 12. In some cases, the regions 261 may be pre-stretched in a manner similar to angioplasty balloons.

In addition to including features at a distal portion of the device sheath, as is shown in FIGS. 20-24, a device sheath 334 may be formed in a folded manner, at its distal end, to allow for radial expansion and contraction as needed. FIGS. 25a and 25b show side-views of such device sheath 334 in the radially unexpanded and radially expanded states, respectively. FIGS. 26a and 26b show end-on views of such device sheath 334 in the radially unexpanded and radially expanded states, respectively. Device sheath 334 is discussed in the context of a closure device that lacks an anchor and a plug, but it will be understood that expandable device sheath 334 may be used with an anchor/plug system such as those discussed above.

In some cases, the device sheath 334 may be introduced using a needle and a guidewire in a conventional manner, except that the device sheath 334 may be delivered in a smaller, folded configuration to simplify its introduction. In some cases, device sheath 334 may include longitudinal reinforcing fibers or wires that may enhance the axial stiffness of device sheath 334 without significantly increasing its delivery profile. FIGS. 26a and 26b are end-on views of device sheath 334 in folded and unfolded configurations, respectively. The device sheath 334 may be introduced over a guidewire 361, or, alternatively, over a needle or dilator.

The guidewire 361 may be removed, and the device sheath 334 may be left in place within the tissue tract or wound. A standard sheath 362 may then be introduced inside device sheath 334 and advanced into the vessel 363. The standard sheath 362 may be used in a conventional manner, for introducing one or more intravascular devices for diagnosis and/or treatment within the vessel 363. After the intravascular treatment, the intravascular diagnosis and/or treatment devices are removed. A hemostasis balloon 364 is then introduced through the standard sheath 362 and inflated to control bleeding. Once the hemostasis balloon 364 is inflated, the standard sheath 362 may be withdrawn. Once the standard sheath 362 is withdrawn, the hemostasis balloon 364 may be slowly deflated over time, allowing stable clot formation. The adjacent tissue may relax and contract, which may in turn contract the device sheath 334.

In the context of the plug/anchor devices discussed above, the sheaths 34, 134, 234 may optionally include folds, as shown in device sheath 334 of FIGS. 25a, 25b, 26a and 26b. In some cases, device sheath 334 may be inserted over or under the sheaths 60, 160, 260.

Although shown herein in the context of vascular sealing, the device sheath disclosed may have additional uses. These may include, for example, therapeutic, diagnostic or implantable device placement.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

What is claimed is:

1. A vascular closure device, comprising:
an insertion sheath having a lumen therethrough;
a device sheath having a distal end capable of being inserted into the lumen of the insertion sheath, the device sheath expandable at or near its distal end;
a handle extending from a proximal end of the device sheath, the handle capable of being coupled to the insertion sheath;
an expandable vascular plug disposed within the distal end of the device sheath; and
wherein the device sheath expands at or near its distal end as the vascular plug is expanded.

2. The vascular closure device of claim 1, wherein the device sheath includes openings proximate the distal portion of the device sheath, the openings are selectively openable such that when the device sheath is extended out of a distal end of the insertion sheath, the vascular plug expands outward from a compressed state against the distal end of the device sheath.

3. The vascular closure device of claim 2, wherein the openings on the device sheath include a material that is capable of softening when exposed to selected conditions.

4. The vascular closure device of claim 2, wherein the openings on the device sheath include a material that is capable of dissolving when exposed to selected conditions.

5. The vascular closure device of claim 2, wherein the openings on the device sheath comprise slits that are generally aligned with a longitudinal axis of the device sheath.

6. The vascular closure device of claim 5, wherein the slits on the device sheath are evenly spaced around the circumference of the device sheath.

7. The vascular closure device of claim 2, wherein the openings on the device sheath comprise longitudinal slots.

8. The vascular closure device of claim 7, wherein the slots on the device sheath are evenly spaced around the circumference of the device sheath.

9. The vascular closure device of claim 7, wherein the slots have a length and a width and the width varies along the length.

10. The vascular closure device of claim 9, wherein the width of the slots decrease along the length and toward the distal end of the device sheath.

11. The vascular closure device of claim 1, wherein the openings on the device sheath comprise intermittent perforations that are generally aligned with a longitudinal axis of the device sheath.

12. The vascular closure device of claim 11, wherein the intermittent perforations on the device sheath are selectively openable with expansion of the vascular plug.

13. The vascular closure device of claim 1, wherein the device sheath comprise score lines proximate the distal portion of the device sheath, the score lines are generally aligned with a longitudinal axis of the device sheath.

14. The vascular closure device of claim 13, wherein the score lines on the device sheath are selectively openable with expansion of the vascular plug.

15. A vascular closure device, comprising:
an insertion sheath having a lumen therethrough;
a device sheath having a distal end capable of being inserted into the lumen of the insertion sheath, the device sheath including a plurality of openings at or near its distal end, the device sheath comprising a first material and including portions of a second material that are evenly spaced around the circumference of the device sheath, the second material being expandable;
a handle extending from a proximal end of the device sheath, the handle capable of being coupled to the insertion sheath;
an anchor disposed at the distal end of the device sheath;
an expandable vascular plug disposed within the distal end of the device sheath; and
a suture operatively connecting the vascular plug to the anchor.

16. The vascular closure device of claim 15, wherein as the distal end of the device sheath is extended out of a distal end of the insertion sheath, the vascular plug expands radially outward from a compressed state against the distal end of the device sheath and expands the second material.

17. The vascular closure device of claim 15, wherein the second material comprises a deformable material.

18. The vascular closure device of claim 15,
wherein the first material has a wall thickness; and
wherein the second material has a wall thickness less than that of the first material.

19. The vascular closure device of claim 15, wherein the second material includes folds that are expandable.

20. A vascular closure system, comprising:
an expandable sheath having at least one longitudinal reinforcement and a first material connected to the reinforcement, the first material being capable of being folded for a delivery configuration and expanded for an expanded configuration, the expandable sheath having an inner lumen capable of being slid over a guidewire or a dilator in the delivery configuration;
an insertion sheath capable of being disposed within the inner lumen of the expandable sheath while in the expanded configuration;
a hemostasis balloon catheter capable of being disposed within the insertion sheath in a deflated configuration; and
a hemostasis balloon disposed on a distal end of the hemostasis balloon catheter, the hemostasis balloon capable of extending out a distal end of the insertion sheath in an inflated configuration.
